(19) United States
(12) Patent Application Publication
(10) Pub. No.: US 2020/0009226 A1
(43) Pub. Date: Jan. 9, 2020

(54) Compositions and Methods for Treating Stroke

(71) Applicant: CardioVascular BioTherapeutics, Inc., Dallas, TX (US)

(72) Inventors: Laurence R. Meyerson, Henderson, NV (US); John J. Jacobs, Berkley, CA (US); Thomas J. Stegmann, Petersberg (DE)

(21) Appl. No.: 16/502,263

(22) Filed: Jul. 3, 2019

Related U.S. Application Data
(60) Provisional application No. 62/693,600, filed on Jul. 3, 2018.

(51) Int. Cl. A61K 38/18; A61K 48/00; A61K 9/00; A61P 43/00

(52) U.S. Cl. CPC A61K 38/1825; A61P 43/00; A61K 9/0019; A61K 48/005

(57) ABSTRACT

The present invention includes a method of treating ischemic stroke, comprising administering to a subject with ischemic stroke an FGF-1, FGF-1₁₋₁₅₅, FGF-1₁₋₁₄₁, or combinations thereof, in an amount sufficient to cross the blood brain barrier and reduce or eliminate the ischemic stroke. In one aspect, the method also includes administering at least one other therapeutic agent to the subject, before, concurrently with or after the FGF-1, FGF-1₁₋₁₅₅, FGF-1₁₋₁₄₁, or combinations thereof.

### CROSS-REFERENCE TO RELATED APPLICATIONS

This application claims priority to U.S. Provisional Application Ser. No. 62/693,600, filed Jul. 3, 2019, the entire contents of which are incorporated herein by reference.

### STATEMENT OF FEDERALLY FUNDED RESEARCH

None.

### TECHNICAL FIELD OF THE INVENTION

The present invention relates in general to the field of compositions and methods for treating stroke.

### BACKGROUND OF THE INVENTION

Without limiting the scope of the invention, its background is described in connection with stroke.

Stroke resulting in brain damage is most often caused by a lack of blood flow to a selected part of the brain. A stroke is characterized by an infracted area of the brain, dead tissue which cannot recover, surrounded by an underperfused area of risk, which would be the target of growth factor treatment. A stroke results in permanent damage to the brain tissue—and in many cases, permanent disability to the patient. Stroke is the third leading cause of death and a leading cause of serious, long-term disability in the United States. The probability of stroke increases as people get older. According to the American Heart Association, approximately 700,000 Americans suffer a stroke each year; about 25% of these strokes are fatal. Stroke is responsible for an estimated $40 billion in health-care costs and lost productivity each year.

Ischemic or occlusive strokes, which account for approximately 80 percent of all strokes, are caused by an obstruction in an artery, generally one of the neck carotid arteries, the major arteries in the neck that carry oxygen-rich blood from the heart to the brain. There is limited treatment available to patients who have suffered a stroke. Thrombolytic therapy using plasminogen activators is sometimes tried in these patients to unblock the arteries supplying blood to the brain, but safety and bleeding issues have prevented this treatment from gaining wide-spread acceptance in the medical community.

Thus, a need remains for novel treatments that can prevent on-going damage to brain tissue after an ischemic stroke, but also to improve brain function following a stroke.

### SUMMARY OF THE INVENTION

In one embodiment, the present invention includes a method of treating ischemic stroke, comprising administering to a subject with ischemic stroke an FGF-1, FGF-1₁₋₁₅₅, FGF-1₁₋₁₄₁, or combinations thereof, in an amount sufficient to cross the blood brain barrier and treat the ischemic stroke. In one aspect, the method further comprises administering at least one other therapeutic agent to the subject, before, concurrently with or after the FGF-1, FGF-1₁₋₁₅₅, FGF-1₁₋₁₄₁, or combinations thereof. In another aspect, the therapeutic agent is selected from the group consisting of a second antibody, a second antibody fragment, an immunoconjugate, an immunomodulator, an anti-angiogenic agent, a pro-apoptotic agent, a cytokine, a chemokine, a drug, a hormone, an siRNA, a coagulation inhibitor, a stem cell growth factor, a lymphotoxin, a hematopoietic factor, a colony stimulating factor, an interferon, erythropoietin, thrombopoietin, an enzyme, recombinant human thrombomodulin and activated human protein C. In another aspect, the FGF-1, FGF-1₁₋₁₅₅, FGF-1₁₋₁₄₁, or combinations thereof, are provided intravenously, subcutaneously, intranasally, stereotaxically delivered into a brain parenchyma, into the cerebrospinal fluid, or in an indwelling Ommaya reservoir. In another aspect, a brain image of the subject is captured in an ambulance and administration of the FGF-1, FGF-1₁₋₁₅₅, FGF-1₁₋₁₄₁, or combinations thereof, occurs before arrival to a hospital. In another aspect, the subject has been administered tissue plasminogen activator after the subject has suffered the cerebral ischemia/reperfusion injury. In another aspect, the FGF-1, FGF-1₁₋₁₅₅, FGF-1₁₋₁₄₁, or combinations thereof, is comprised within a pharmaceutical composition formulated for injection, or for sustained release. In another aspect, the FGF-1, FGF-1₁₋₁₅₅, FGF-1₁₋₁₄₁, or combinations thereof, is administered to the subject within 24 hrs of the onset of symptoms of ischemic stroke. In another aspect, the FGF-1, FGF-1₁₋₁₅₅, FGF-1₁₋₁₄₁, or combinations thereof, is repeatedly administered to the subject at least once per day for at least 3 days. In another aspect, the FGF-1, FGF-1₁₋₁₅₅, FGF-1₁₋₁₄₁, or combinations thereof, is repeatedly administered to the subject for at least 7 days. In another aspect, the FGF-1, FGF-1₁₋₁₅₅, FGF-1₁₋₁₄₁, or combinations thereof, is provided at 20, 50, 100, 200, 500 and 1000 ug/kg/hr. In another aspect, the FGF-1, FGF-1₁₋₁₅₅, FGF-1₁₋₁₄₁, or combinations thereof, reduces an infarct volume by more than 25, 30, 33, 35, 40, 45, or 50% when compared to a non-treated tissue. In another aspect, the FGF-1, FGF-1₁₋₁₅₅, FGF-1₁₋₁₄₁, or combinations thereof, reduces a neurological deficit by at least 30, 40, 50, 60, or 70% when compared to a non-treated tissue. In another aspect, a first injection site is within the ischemic region of brain tissue. In another aspect, a first injection site is directly adjacent to the ischemic region of brain tissue. In another aspect, a first injection site is outside of the ischemic region of brain tissue. In another aspect, the method further comprises: obtaining a preoperative non-invasive image data of the subject, the preoperative non-invasive image data including a region of brain tissue, analyzing the preoperative non-invasive image data to preoperatively identify at least one at-risk region of brain tissue, preoperatively identifying at least one abnormality within a blood vessel supply to the at-risk region of brain tissue, operatively administering a therapeutically effective amount of the FGF-1, FGF-1₁₋₁₅₅, FGF-1₁₋₁₄₁, or combinations thereof, wherein the administration of the FGF-1, FGF-1₁₋₁₅₅, FGF-1₁₋₁₄₁, or combinations thereof induces growth of supplemental blood vessels proximate to the abnormality.

In another embodiment, the present invention includes a method of treating ischemic stroke, comprising: identifying a subject in need of treatment for ischemic stroke; and administering to a subject with ischemic stroke an FGF-1, FGF-1₁₋₁₅₅, FGF-1₁₋₁₄₁, or combinations thereof, in an amount sufficient to cross the blood-brain barrier. In one aspect, the method further comprises administering at least one other therapeutic agent to the subject, before, concurrently with or after the FGF-1, FGF-1₁₋₁₅₅, FGF-1₁₋₁₄₁, or combinations thereof. In another aspect, the therapeutic agent is selected from the group consisting of a second antibody, a second antibody fragment, an immunoconjugate, an immunomodulator, an anti-angiogenic agent, a pro-apoptotic agent, a cytokine, a chemokine, a drug, a hormone, an siRNA, a coagulation inhibitor, a stem cell growth factor, a lymphotoxin, a hematopoietic factor, a colony stimulating factor, an interferon, erythropoietin, thrombopoietin, an enzyme, recombinant human thrombomodulin and activated human protein C. In another aspect, the FGF-1, FGF-1₁₋₁₅₅, FGF-1₁₋₁₄₁, or combinations thereof, are provided intravenously, subcutaneously, intranasally, stereotaxically delivered into a brain parenchyma, into the cerebrospinal fluid, or in an indwelling Ommaya reservoir. In another aspect, a brain image of the subject is captured in an ambulance and administration of the FGF-1, FGF-1₁₋₁₅₅, FGF-1₁₋₁₄₁, or combinations thereof, occurs before arrival to a hospital. In another aspect, the subject has been administered tissue plasminogen activator after the subject has suffered the cerebral ischemia/reperfusion injury. In another aspect, the FGF-1, FGF-1₁₋₁₅₅, FGF-1₁₋₁₄₁, or combinations thereof, is comprised within a pharmaceutical composition formulated for injection or sustained release. In another aspect, the FGF-1, FGF-1₁₋₁₅₅, FGF-1₁₋₁₄₁, or combinations thereof, is administered to the subject within 24 hrs of the onset of symptoms of ischemic stroke. In another aspect, the FGF-1, FGF-1₁₋₁₅₅, FGF-1₁₋₁₄₁, or combinations thereof, is repeatedly administered to the subject at least once per day for at least 3 days. In another aspect, the FGF-1, FGF-1₁₋₁₅₅, FGF-1₁₋₁₄₁, or combinations thereof, is repeatedly administered to the subject for at least 7 days. In another aspect, the FGF-1, FGF-1₁₋₁₅₅, FGF-1₁₋₁₄₁, or combinations thereof, is provided at 20, 50, 100, 200, 500 and 1000 ug/kg/hr. In another aspect, the FGF-1, FGF-1₁₋₁₅₅, FGF-1₁₋₁₄₁, or combinations thereof, reduces an infarct volume by more than 25, 30, 33, 35, 40, 45, or 50% when compared to a non-treated tissue. In another aspect, the FGF-1, FGF-1₁₋₁₅₅, FGF-1₁₋₁₄₁, or combinations thereof, reduces a neurological deficit by at least 30, 40, 50, 60, or 70% when compared to a non-treated tissue. In another aspect, the method further comprises: obtaining a preoperative non-invasive image data of the subject, the preoperative non-invasive image data including a region of brain tissue, analyzing the preoperative non-invasive image data to preoperatively identify at least one at-risk region of brain tissue, preoperatively identifying at least one abnormality within a blood vessel supply to the at-risk region of brain tissue, operatively administering a therapeutically effective amount of the FGF-1, FGF-1₁₋₁₅₅, FGF-1₁₋₁₄₁, or combinations thereof, wherein the administration of the FGF-1, FGF-1₁₋₁₅₅, FGF-1₁₋₁₄₁, or combinations thereof induces growth of supplemental blood vessels proximate to the abnormality. In another aspect, the method further comprises administering the FGF-1, FGF-1₁₋₁₅₅, FGF-1₁₋₁₄₁, or combinations thereof to induce growth of blood vessels proximate to the abnormality, wherein at least a portion of the blood vessel supply to the at-risk region of brain tissue is redirected through the supplemental blood vessels. In another aspect, the abnormality is located within the at-risk region of brain tissue. In another aspect, the abnormality is located within the region of brain tissue. In another aspect, the method further comprises obtaining postoperative non-invasive image data of the subject, the post-operative non-invasive image data including the at-risk region of brain tissue, analyzing the post-operative non-invasive image data to identify any improvement in the blood vessel supply to the at-risk region of brain tissue. In another aspect, a first injection site is within the ischemic region of brain tissue. In another aspect, a first injection site is directly adjacent to the ischemic region of brain tissue. In another aspect, a first injection site is outside of the ischemic region of brain tissue.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the effect of FGF-1₁₋₁₅₁ on infarct volumes in the mouse following transient ischemia. All mice were subjected to 1 hr of cerebral ischemia followed by 24/48 hrs of reperfusion. Animals were infused with vehicle (control) or FGF-1 intravenously at the indicated times (hrs) following ischemia. Animals were sacrificed at 24 or 48 hrs and processed to determine the infarct volume. p<0.0001 for FGF-1 (1, 2, and 4 hr) compared to Vehicle.

FIG. 2 is a graph that shows the effect of FGF-1₁₋₁₅₁ on neurological deficits in the mouse following transient ischemia. All mice were subjected to 1 hr of cerebral ischemia followed by 24/48 hrs of reperfusion and initiation of FGF-1 treatment at the times indicated. Animals were examined for neurological deficits at 22/46 hrs after ischemia. p<0.0001 for each treated group compared to control.

FIG. 3 is a graph that shows the effect of FGF-1₁₋₁₅₁ on cerebral blood flow in the mouse following transient ischemia. All mice were subjected to 1 hr of cerebral ischemia followed by 24/48 hrs of reperfusion and initiation of FGF-1 treatment at the times indicated. Animals were examined for CBF at 10 min prior to, during and after ischemia. p=NS.

FIG. 4 is a graph that shows the effect of FGF-1₁₋₁₅₁ on blood pressure in the mouse following transient ischemia. All mice were subjected to 1 hr of cerebral ischemia followed by 24/48 hrs of reperfusion and initiation of FGF-1 treatment at the times indicated. Animals were examined for BP at 10 min prior to, during and after ischemia. p=NS.

FIG. 5 is a graph that shows the effect of FGF-1₁₋₁₅₁ on heart rate in the mouse following transient ischemia. All mice were subjected to 1 hr of cerebral ischemia followed by 24/48 hrs of reperfusion and initiation of FGF-1 treatment at the times indicated. Animals were examined for HR at 10 min prior to, during and after ischemia. p=NS.

FIG. 6 is a graph that shows the effect of FGF-1₁₋₁₅₁ on pH changes in blood in the mouse following transient ischemia. All mice were subjected to 1 hr of cerebral ischemia followed by 24/48 hrs of reperfusion and initiation of FGF-1 treatment at the times indicated. Animals were examined for pH at 10 min prior to, during and after ischemia. p=NS.

FIG. 7 is a graph that shows the effect of FGF-1₁₋₁₅₁ on pO2 changes in blood in the mouse following transient ischemia. All mice were subjected to 1 hr of cerebral ischemia followed by 24/48 hrs of reperfusion and initiation of FGF-1 treatment at the times indicated. Animals were examined for pO2 at 10 min prior to, during and after ischemia. p=NS.

FIG. 8 is a graph that shows the effect of FGF-1₁₋₁₅₁ on pCO2 changes in blood in the mouse following transient ischemia. All mice were subjected to 1 hr of cerebral ischemia followed by 24/48 hrs of reperfusion and initiation of FGF-1 treatment at the times indicated. Animals were examined for pCO2 at 10 min prior to, during and after ischemia. p=NS.

FIG. 9 is a graph that shows the results from a study in mice with experimental stroke: Effect of FGF-1₁₋₁₅₁ on Infarct Volumes in the Mouse Following Transient Ischemia. All mice were subjected to 1 hr of cerebral ischemia followed by 24 hrs of reperfusion. Animals were infused with vehicle (control), FGF-1₁₋₁₅₁ or FGF-2 intravenously at the end of ischemia for 3 hrs. Animals were sacrificed on day 2 and processed to determine the infarct volume. Similar results were obtained with FGF-1₁₋₁₄₁. FGF-2, or basic FGF, is another member of the fibroblast growth factor family and has previously been shown to be efficacious in this animal model of stroke.

FIG. 10 is a graph that shows the results from a study in mice with experimental stroke: Effect of FGF-1₁₋₁₅₁ on Infarct Volumes in the Mouse Following Transient Ischemia. Similar results were obtained with FGF-1₁₋₁₄₁. All mice were subjected to 1 hr of cerebral ischemia followed by 24 hrs of reperfusion. Animals were infused with vehicle (control), FGF-1₁₋₁₅₁ or FGF-2 intravenously at the end of ischemia for 3 hrs. Animals were sacrificed on day 2 and processed to determine the infarct volume. FGF-2 was not tested in this series of experiments.

FIG. 11 is a graph that shows the effect of FGF-1₁₋₁₅₁ on Neurological Deficits in the Mouse Following Transient Ischemia. Similar results were obtained with FGF-1₁₋₁₄₁. All mice were subjected to 1 hr of cerebral ischemia followed by infusion of FGF-1₁₋₁₅₁. Animals were examined for neurological deficits at 22 hrs after ischemia.

FIG. 12 is a graph that shows the effect of FGF-1 the size of Stroke Volume Correlates with Behavioral Deficits. It can be seen that there is a very tight correlation between the size of the stroke volume and the degree of behavioral deficits in mice given a stroke. By reducing the volume of stroke, FGF-1₁₋₁₅₁ significantly reduces behavioral deficits.

FIG. 13 is a graph that shows the effect of FGF-1₁₋₁₅₁ on cerebral blood flow in the mouse following transient ischemia. Similar results were obtained with FGF-1₁₋₁₄₁. All mice were subjected to 1 hr of cerebral ischemia followed by 24 hrs of reperfusion. Animals were examined for CBF at 10 min prior to, during and after ischemia.

FIG. 14 is a graph that shows the effect of FGF-1₁₋₁₅₁ on blood pressure in the mouse following transient ischemia. Similar results were obtained with FGF-1₁₋₁₄₁. All mice were subjected to 1 hr of cerebral ischemia followed by 24 hrs of reperfusion. Animals were examined for BP at 10 min prior to, during and after ischemia.

FIG. 15 is a graph that shows the effect of FGF-1₁₋₁₅₁ on heart rate in the mouse following transient ischemia. Similar results were obtained with FGF-1₁₋₁₄₁. All mice were subjected to 1 hr of cerebral ischemia followed by 24 hrs of reperfusion. Animals were examined for HR at 10 min prior to, during and after ischemia.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Arteriogenesis is technically considered remodeling of pre-existing vascular channels (collaterals) or de novo artery formation, it can be stimulated by local changes in perfusion (shear stress), as well as cellular influx and proliferation, and associated with a 20-30 fold increase in blood flow. Vasculogenesis is technically considered on the one hand to encompass embryonic vascular development, and on the other hand to include de novo formation or remodeling of pre-existing vascular channels initiated by circulating vascular precursor cells; furthermore; it is considered to be ischemia and injury initiated. The term “angiogenesis” is meant to encompass all three technical terms.

Angiogenesis is known to occur physiologically during zygote implantation, embryogenesis, post-embryonic growth, and during tissue repair and remodeling. Pathologically, uncontrolled angiogenesis is associated with a variety of diseases such as macular degeneration, diabetic retinopathy, inflammation, including arthritis and psoriasis, and cancer. One common aspect of adult angiogenesis is tissue hypoxia. In situations of tissue expansion, cells are typically dependent on the microvasculature for nutrients and oxygen supply, as well as removal of metabolic waste products. Accordingly, during tissue growth, cells begin to “sense” a lack of oxygen. This triggers a cascade of events that culminates in angiogenesis. During pathological conditions, such as the conditions associated with hypoxic and/or ischemic disc disease, the lack of oxygen is induced through hypoperfusion. Said hypoperfusion may occur due to, for example, atherosclerosis. In some pathological conditions, the normal angiogenic response to hypoxia is absent or substantially diminished.

As used herein, the terms “treating,” “treatment,” “therapeutic,” or “therapy” do not necessarily mean total cure or abolition of the disease or condition, but rather, include any alleviation of any undesired signs or symptoms of a disease or condition, to any extent, can be considered treatment and/or therapy. It is entirely possible that “treatment” consists of a temporary improvement of the endplate vasculature that requires repeated treatment over time to continue the regenerative process. Furthermore, treatment may include acts that may worsen the patient's overall feeling of well-being or appearance.

As used herein, the phrase “therapeutically effective amount” refers to a compound as used herein to indicate an amount of an active compound, or pharmaceutical agent, that elicits the biological or medicinal response indicated, e.g., FGF-1. This response may occur in a tissue, system, animal or human and includes alleviation of the symptoms of the disease being treated. The exact formulation, route of administration and dosage for the composition and pharmaceutical compositions disclosed herein can be chosen by the individual physician in view of the patient's condition. (See e.g., Fingl et al. 1975, in “The Pharmacological Basis of Therapeutics”, Chapter 1, and updates thereof, or Remington's Pharmaceutical Sciences, Mace Publishing Company, Philadelphia, Pa., 17th ed. (1985) and updates thereof, relevant portions incorporated herein by reference). Therapeutic treatments can be achieved with small molecule organic drugs or biologics, such as proteins. Typically, the dose range of a small molecule therapeutic agent is administered from about 0.5 to 1000 μg/kg, or 1 to 500 μg/kg, or 10 to 500 μg/kg, or 50 to 100 μg/kg of the patient's body weight per dose. The dose of a therapeutic protein growth factor, such as truncated forms of FGF-1, can be administered to the patient intravenously or intraarterially as either a bolus dose or by infusion from about 0.1 to 100 μg/kg of the patient's body weight, or 0.3 to 30 μg/kg, or 1 to 3 μg/kg of the patient's body weight per dose. To achieve localized targeted dosing, FGF-1 can be injected either directly into or adjacent to the ischemic vertebral endplate, preferably either into or as near as practical to the region of ischemia. Localized dose ranges can be from 10 ng/cm³ to 1 mg/cm³, or 100 ug/cm³ to 100 ug/cm³ or 1 ug/cm³ to 10 ug/cm³ of target vertebral endplate tissue per dose. Local doses can be administered at each ischemic endplate. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

The pharmaceutical composition(s) of FGF-1₁₋₁₅₅, the FGF-1₁₋₁₄₁, or both may be formulated for injection and administered by injection, e.g., intraperitoneal, intramuscular, or intravenous injection. Such compositions can have a pH of between 6.5 and 8.5 or between 6.8 and 7.8. Excipients/carriers/other ingredients can include a sterile aqueous buffer, an isotonizing agent, a microbicidal agent or preservative, a chelating agent, a solubility enhancing agent such as dimethylsulfoxide, and/or other ingredients. The isotonizing agent can be, e.g., sorbitol, glycerine, polyethylene glycol, propylene glycol, glucose and sodium chloride. The microbicidal agent/preservative can be, e.g., para-oxybenzoic acid esters, benzyl alcohol, para-chloro-meta-xylenol, chlorocresol, phenetyl alcohol, sorbic acid and salts thereof, thimerosal, chlorobutanol, etc. The chelating agent can be, for example, sodium edetate, sodium citrate or the sodium salt of condensed phosphoric acid. Other forms of FGF-1 may also be used, e.g., non-natural variants of FGF-1 that are still biologically active (activate FGF-1 receptors), but have a sequence that is not found in nature. For examples, truncated mutant FGF-1 proteins for use with the present invention include: synthetic genes that encode a 140 or 141 amino acid protein of SEQ ID NO:1, which is the mature form of human, also referred to as: FGF-1₁₋₁₄₀, FGF-1₁₋₁₄₁, FGF-1₁₀₋₁₄₀, FGF-1₁₀₋₁₄₁, FGF-1₁₋₁₄₀, FGF-1₁₂₋₁₄₀, FGF-1₁₂₋₁₄₁, and mature FGF-1 with point mutants including, for example, one or more of the following: K9A, K12V, S17R, N18R, N18K, H21Y, R35E, L44F, A66C, Y94V, N95V, H102Y, F108Y, N114R, N114K, C117V, L133A of the mature form of the human FGF-1 (FGF-1₁₆₋₁₅₅). The full length human FGF-1 is UniProtKB—P05230 and its gene sequence (FGF1_HUMAN), Entrez Gene: 2246) are incorporated herein by reference. Mutant FGF-1s with one or more amino acid insertions, deletions or substitutions are introduced by standard genetic engineering techniques, such as site-directed, deletion, and insertion mutagenesis. The wild type FGF-1 three-dimensional conformation is known to be marginally stable with denaturation occurring either at or near physiologic temperature. FGF-1 binding to heparin increases the thermal inactivation temperature by approximately 20° C., thus, in certain embodiments the mutant FGF-1 is combined with heparin. Further, the mutant FGF-1 of the present invention can also be formulated with a therapeutically approved USP heparin, or a mutant heparin. The truncations, insertions, deletions or substitutions of the mutant FGF-1 tends to enhance half-life, which is further enhanced by the inclusion of heparin. Further, mutant heparins can also be used to further enhance the half-life or activity of the mutant FGF-1 used herein. However, heparin is an anti-coagulant that can promote bleeding as a function of increasing concentration. In addition, some individuals have been immunologically sensitized to heparin by previous therapeutic exposure, which can lead to heparin-induced thrombocytopenia and thrombotic events. Mutations that extend the storage stability in vitro and biologic activity in vivo would allow FGF-1 to be formulated and dosed in the absence of exogenous heparin. These include mutations that decrease the rate of oxidative inactivation, such as replacement of one or more of the three cysteine residues by either serine or other compatible residues. In particular, as has been described by others, substitution of cysteine 117 by serine is known to substantially increase the half-life of human FGF-1 by decreasing the rate of oxidative inaction. Other mutations have been described that increase conformational stability by making amino acid changes in internal buried and/or external exposed amino acid residues. In the case of repeat dosing regimens, FGF-1s exhibiting greater stability and life-time might effectively decrease the frequency and number of repeated doses needed to achieve sustained exposure and greater efficacy. These stabilized mutants would allow longer duration dosing from slow release polymeric matrices and delivery systems.

In other embodiments it may be desirable to utilize an angiogenesis-stimulating protein for administration in a therapeutically effective amount. Said protein may be selected from proteins known to stimulate angiogenesis, including but not limited to TPO (thyroid peroxidase), SCF (stem cell factor), IL-1 (interleukin 1), IL-3, IL-6, IL-7, IL-11, flt-3L (fms-like tyrosine kinase 3 ligand), G-CSF (granulocyte-colony stimulating factor), GM-CSF (granulocyte monocyte-colony stimulating factor), Epo (erythropoietin), FGF-1, FGF-2, FGF-4, FGF-5, FGF-20, IGF (insulin-like growth factor), EGF (epidermal growth factor), NGF (nerve growth factor), LIF (leukemia inhibitory factor), PDGF (platelet-derived growth factor), BMPs (bone morphogenetic protein), activin-A, VEGF (vascular endothelial growth factor), VEGF-B, VEGF-C, VEGF-D, PlGF, and HGF (hepatocyte growth factor). In some preferred embodiments, administration of the angiogenesis-stimulating protein is performed by injection directly into a vertebral body. In some embodiments, the angiogenic-stimulating protein is co-administered with stem or progenitor cells.

In some embodiments a carrier solution or containing/metering device may be desired. Appropriate carrier solutions may be selected based on properties such as viscosity, ease of administration, ability to bind solution over a period of time, and general affinity for the agent delivered. Said solutions may be modified or additives incorporated for modification of biological properties. Starting solutions may include certain delivery polymers known to one who is skilled in the art. These could be selected from, for example: polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acryl substituted cellulose acetates, non-degradable polyurethanes, polystyrenes, polyvinyl fluoride, polyvinyl imidazole, chlorosulphonated polyolefin, and polyvinyl alcohol.

Administration may be performed under fluoroscopy or by other means in order to allow for localization in proximity of the cause of hypoperfusion. Acceptable carriers, excipients, or stabilizers are also contemplated within the current invention; said carriers, excipients and stabilizers being relatively nontoxic to recipients at the dosages and concentrations employed, and may include buffers such as phosphate, citrate, and other organic acids; antioxidants including ascorbic acid, n-acetylcysteine, alpha tocopherol, and methionine; preservatives such as hexamethonium chloride; octadecyldimethylbenzyl ammonium chloride; benzalkonium chloride; phenol, benzyl alcohol, or butyl; alkyl parabens such as methyl or propyl paraben; catechol; resorcinol; cyclohexinol; 3-pentanol; and mecresol; low molecular weight polypeptides; proteins, such as gelatin, or non-specific immunoglobulins; amino acids such as glycine, glutamine, asparagine, histidine, arginine, or lysine; monosaccharides, disaccharides, and other carbohydrates including glucose, mannose, or dextrins; chelating agents such as EDTA (ethylenediaminetetraacetic acid); sugars such as sucrose, mannitol, trehalose, or sorbitol; salt-forming counter-ions such as sodium. For heparin-binding proteins, including FGFs, heparin may be incorporated into the formulation, which can bind and stabilize the protein against inactivation and degradation.

In various embodiments, treatment of hypoxic and/or ischemic disc disease could include the use of a biocompatible or biodegradable implant. Said biodegradable implants can contain a biodegradable delivery system, or carrier, as well as angiogenic factors; said angiogenic factors could be capable of stimulating sufficient neovascularization to overcome local hypoxia. One preferred angiogenic factor is fibroblast growth factor 1 (FGF-1). However, other recombinant naturally derived, in vitro derived, and in vivo derived angiogenic factors may also be used. In some embodiments, the biodegradable implant that contains the angiogenic factors contains a carrier. The carrier is preferably chosen so as to remain within the implanted site for a prolonged period and slowly release the angiogenic factors contained therein to the surrounding environment. This mode of delivery allows said angiogenic factors to remain in therapeutically effective amounts within the site for a prolonged period. By providing said angiogenic factors within a carrier, the advantage of releasing said angiogenic factors directly into the target area is realized. In some embodiments, the implant's carrier is provided in an injectable form. Injectability allows the carrier to be delivered in a minimally invasive and preferably percutaneous method. In some embodiments, the injectable carrier is a gel. In others, the injectable carrier comprises hyaluronic acid (HA).

In various embodiments, angiogenic treatments can be used in conjunction with other treatments, such as introduction and/or injection of stem cells, which may be embryonic stem cells or adult stem cells. Such angiogenic treatments could be used to prepare tissues for subsequent injection of stem cells, or angiogenic compounds could be injected concurrently with and/or after introduction of such cells. With regards to intervertebral disc tissues or other tissues, growth factors, synthetic or treated allograft or xenograft tissue for scaffold (or extra-cellular matrix) and stem cells (each of which could be used separately or in varying levels of in combination with each other) could be utilized to “engineer” or otherwise modify disc tissue with the goal of regenerating living tissue within the intervertebral disc. If the degenerative disc to be treated required that ischemia or hypoxia related causes needed to be diagnosed and treated first or in combination with the tissue engineering techniques (or if such treatment could be optimized if such approaches were employed), then the diagnosis and treatment could be for ischemic disc disease or other pathologies such as described herein.

In addition, it may be determined that a combination of stem cells, engineered tissue, scaffold, growth factors, or combinations thereof, would be enhanced by combining angiogenic factors such as FGF-1 in its native state or through an FGF-1 mutant (through protein engineering technology) or any other appropriate angiogenic factor to decrease the stroke area.

In certain embodiments, a preoperative planning is desirable to map the areas to be treated, if the therapeutic agent in intracranially injected. Preoperative imaging, as described before, could analyze the metabolic demands of the combination transplant and the state of the nutrient pathway that is required to support the transplant. Detailed preoperative planning, using imaging modalities already discussed (or imaging modalities not yet invented or used for this type of procedure) of the nutrient demands of the transplant and the subsequent translation of this imaging data into the proper amount, delivery, vehicle, approach, and area of ischemia of the stroke.

One main dysfunction associated with ischemic brain disease appears to be a loss of perfusion of oxygenated blood to the heart tissue. If stem cell, gene therapy, protein therapy, tissue therapy or any combination thereof were implanted at or within brain tissue and/or otherwise directed towards the tissue of the brain, the metabolic demands of that transplant could be calculated with preoperative imaging and the proper angiogenic treatment delivered based upon that calculation. Alternatively, if the imaging demonstrated a range of breakdown of the delivery pathway to the brain tissue, cells, proteins, genes or any combination thereof, then a more non-specific dose of angiogenic therapy might be desired. The angiogenic treatment could be initiated, based on brain imaging data, prior to the regenerative treatment so that angiogenesis would already be present when the transplant is performed. In addition, the angiogenic treatment could be combined with the tissue/cell/signal transplant (or other regenerative embodiment), providing brain capillary growth and nutrient delivery to enhance healing of the transplant at the time of the procedure or subsequently after surgery. Administration of such factors could be accomplished prior to, during and/or after such brain surgery to the patient, as desired.

### Example 1. Treatment of Ischemic Brain Disease with FGF-1

FGF-1 was tested for efficacy in the middle cerebral artery occlusion (MCAO) model in the mouse. Intravenous (i.v., 3 hrs) infusion of FGF-1 starting at 1, 2, 4, 8, 12 or 24 hrs after the initiation of MCAO was compared to i.v. infusion of vehicle. Brains were excised and stained with triphenyltetrazolium chloride (TTC) and examined for infarct volume by image analysis. Infusion of FGF-1 demonstrated a time dependent effect in reducing the infarct volume following ischemic injury. FGF-1 showed an 86% reduction in infarct volume at 1000 μg/kg at 1 hr following the initiation of ischemia. This was statistically significant at p<0.0001, indicating that there was a significant protection for ischemic injury. FGF-1 was effective at 1000 μg/kg when initiated at 2, and 4 hrs after ischemic injury (79%, and 64% reduction in infarct volume, respectively). There was a reduction in infarct volume at 8 hrs after initiation of ischemia however was at the borderline of significance. Overall, FGF-1 at 1000 μg/kg was the effective at limiting the extent of MCAO in the brain following induction of ischemia/reperfusion in the mouse out to 8 hrs.

Study design. The mouse model of stroke, middle cerebral artery occlusion (MCAO). Mice were subjected to 1 hr MCAO followed by 24 or 48 hrs of reperfusion. Vehicle or FGF-1 were infused at various times (1, 2, 4, 8, 12 or 24 hrs) at the end of ischemia for three hrs and on the second day mice were sacrificed and examined for infarct volume by TTC staining.

In Vivo methods. Male C57BL/6 (Jackson Laboratory) mice weighing approximately 25 grams each were given free access to food and water before and during the experiment. Animals were acclimated for 1 week prior to experimentation. The animals were infused with vehicle or FGF-1 (0 or 1000 μg/kg/hr). Mice were infused intravenously for 3 hrs, at 1, 2, 4, 8, 12 or 24 hrs after the initiation of ischemia.

Induction of ischemia. Each mouse was subjected to one hr of cerebral ischemia followed by 24 hrs of reperfusion. At the end of the ischemic period, animals were infused with vehicle or FGF-1 at the indicated times and at 24 or 48 hrs examined for infarct volume. Each mouse was anesthetized and a thermistor probe was inserted into the rectum to monitor body temperature, which was maintained at 36-37° C. by external warming. The left common carotid artery (CCA) was exposed through a midline incision in the neck. The superior thyroid and occipital arteries were electrocoagulated and divided. A microsurgical clip was placed around the origin of the internal carotid artery (ICA). The distal end of the ECA was ligated with 6-0 silk and transected. A 6-0 silk is tied loosely around the ECA stump. The clip is removed and the fire-polished tip of a 5-0 nylon suture (poly-L-lysine coated) was gently inserted into the ECA stump. The loop of the 6-0 silk was tightened around the stump and the nylon suture was advanced approximately 11 mm (adjusted for body weight) into and through the internal carotid artery (ICA) after removal of the aneurysm clip, until it rested in the anterior cerebral artery (ACA), thereby occluding the anterior communicating and middle cerebral arteries. The animal was returned to home cage after removal from anesthesia. After the nylon suture had been in place for 1 hr, the animal was re-anesthetized, rectal temperature was recorded, the suture was removed, and the incision closed.

Infarct volume determination. For infarct volume determination, the animals were anesthetized with an intraperitoneal injection of sodium pentobarbital (50 mg/kg). The brains were removed, sectioned into 4 2-mm sections through the infracted region and placed in 2% triphenyltetrazolium chloride (TTC) for 30 minutes at 24 or 48 hrs. After, the sections were placed in 4% paraformaldehyde overnight. The infarct area in each section was determined with a computer-assisted image analysis system, consisting of a Power Macintosh computer equipped with a Quick Capture frame grabber card, Hitachi CCD camera mounted on a camera stand. NIH Image Analysis Software, v. 1.55 was used. The images were captured, and the total area of infarct was determined over the sections. A single operator blinded to treatment status performed all measurements. Summing the infarct volumes of the sections calculated the total infarct volume.

Behavioral analysis. Neurological deficits were assessed 22 or 46 hrs after ischemia based on a scale from 0 (no deficits) to 4 (severe deficits) as described previously (Huang et al., 1994). Neurological scores are as follows: 0, normal motor function; 1, flexion of torso and contralateral forelimb when animal is lifted by the tail; 2, circling to the contralateral side when held by the tail on a flat surface, but normal posture at rest; 3, leaning to the contralateral side at rest; 4, no spontaneous activity.

Cerebral blood flow analysis. CBF was determined in anesthetized animals under resting conditions, 30 minutes into the ischemia, and 10 minutes after reperfusion. Measurements were made using a laser Doppler flowmeter (Moor Instruments). Two flexible probe tips were secured 2 mm posterior and 3 mm lateral to the bregma and 2 mm posterior (peri-infarct region) and 6 mm lateral to the bregma on the ischemic hemisphere.

Physiological parameters. Arterial blood samples (50 μl) were analyzed for pH, arterial oxygen pressure, and partial pressure of carbon dioxide using a blood gas/pH analyzer (Heska iStat-200 analyzer). Samples were taken immediately before, 10 min after MCA occlusion, and 10 mice after the start of reperfusion. Rectal and temporalis muscle temperature was maintained at 37 C. Blood pressure and heart rate were determined using a Visi-System blood pressure monitor.

Statistical analysis. The results are expressed as the mean±standard deviation (SD). The significance of difference in the infarct volume data was analyzed using a t-test.

Exclusion of Animals from the Study. Animals will be excluded from the study based upon several criteria:

1. Animals that died prior to completion of study (at any point).

2. Animals developed severe complications following injection of test articles.

Treatment groups. All groups were subjected to MCAO. Animals (129 animals) were subjected to infusion with vehicle or FGF-1 following MCAO.

Mouse Stroke Model:

| | | Com- | | |
|---|---|---|---|---|
| Group | | pound | Dose (mg/kg) | Route |
| 1 | (n = 10 mice) | Vehicle | 0 ug/kg/hr × 3 hrs start at 1 hr | IF |
| 2 | (n = 10 mice) | Vehicle | 0 ug/kg/hr × 3 hrs start at 2 hr | IF |
| 3 | (n = 10 mice) | Vehicle | 0 ug/kg/hr × 3 hrs start at 4 hr | IF |
| 4 | (n = 10 mice) | Vehicle | 0 ug/kg/hr × 3 hrs start at 8 hr | IF |
| 5 | (n = 10 mice) | Vehicle | 0 ug/kg/hr × 3 hrs start at 12 hr | IF |
| 6 | (n = 10 mice) | Vehicle | 0 ug/kg/hr × 3 hrs start at 24 hr | IF |
| 7 | (n = 10 mice) | FGF-1 | 1000 ug/kg/hr × 3 hrs start at 1 hr | IF |
| 8 | (n = 10 mice) | FGF-1 | 1000 ug/kg/hr × 3 hrs start at 2 hr | IF |
| 9 | (n = 10 mice) | FGF-1 | 1000 ug/kg/hr × 3 hrs start at 4 hr | IF |
| 10 | (n = 10 mice) | FGF-1 | 1000 ug/kg/hr × 3 hrs start at 8 hr | IF |
| 11 | (n = 10 mice) | FGF-1 | 1000 ug/kg/hr × 3 hrs start at 12 hr | IF |
| 12 | (n = 10 mice) | FGF-1 | 1000 ug/kg/hr × 3 hrs start at 24 hr | IF |

Endpoints: Infarct volume in the brain.

All test groups (groups 1-12) including vehicle have been provided to NTS as lyophilized powder.

All animals in the test groups were dosed as indicated above.

At the end of the study, animals were sacrificed for TTC staining and infarct analysis.

Ischemia in mice. The relative severity of ischemia in these studies was assessed. Data was collected from mice with ischemic injury that were intravenously infused with vehicle or FGF-1.

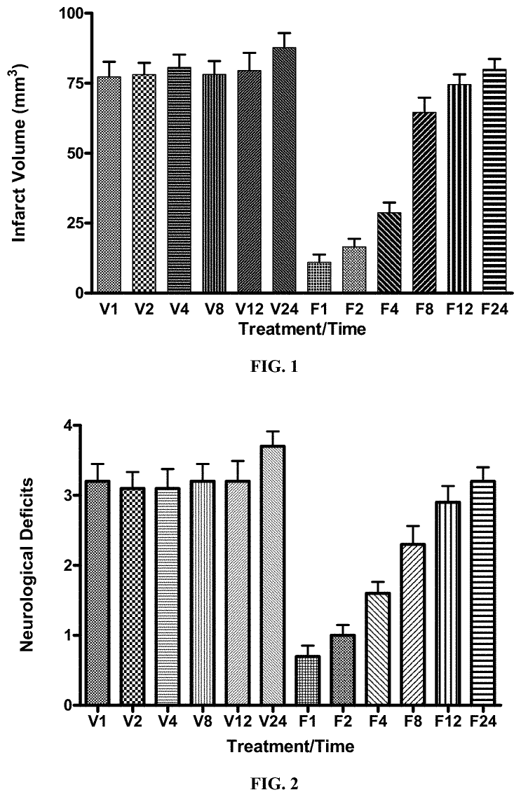

Infarct Area: Compared with the vehicle-infused group, the infarct area in the brains was significantly decreased with some of the FGF-1 groups. FGF-1 showed a time dependent reduction in infarct volume from 1 to 24 hr at 1000 μg/kg/hr (Table 1). Infarct volumes are plotted in FIG. 1. The percent decrease and P value in infarct volume present in the brains are presented in Table 1. As shown in the table, FGF-1 at 1000 μg/kg/hr showed an 86 (1 hr), 79 (2 hr), 64 (4 hr), 17 (8 hr) and 6% (12 hr) decrease in infarct volume compared to vehicle, respectively. However, only the 1, 2, and 4 hr groups showed a significant decrease compared to the vehicle treated groups. In addition, the 24 hr FGF-1 treated animals showed a decrease over the 24 vehicle treated animals (analysis performed at 48 hrs). There was no significant difference compared to the 24 hr vehicle group.

TABLE 1
Percent decrease in infarct in the brain.

| | | | | Time of | | Infarct volume | % reduction in | |
|---|---|---|---|---|---|---|---|---|
| Group | Dose | | ID | infusion | | (mm3 ± SD) | Infarct volume | P- value |
| 1 | 0 | ug | Vehicle | 1 | hr | 77.30 ± 17.01 | 0 | NS |
| 2 | 0 | ug | Vehicle | 2 | hr | 78.09 ± 13.23 | +101%* | NS |
| 3 | 0 | ug | Vehicle | 4 | hr | 80.64 ± 14.46 | +104%* | NS |
| 4 | 0 | ug | Vehicle | 8 | hr | 78.18 ± 14.92 | +101%* | NS |
| 5 | 0 | ug | Vehicle | 12 | hr | 79.54 ± 19.97 | +102%* | NS |
| 6 | 0 | ug | Vehicle | 24 | hr | 87.74 ± 16.31 | +114%* | NS (0.1784) |
| 7 | 1000 | ug | FGF1 | 1 | hr | 11.02 ± 8.711 | −85.7%** | 0.0001 |
| 8 | 1000 | ug | FGF1 | 2 | hr | 16.53 ± 9.180 | −78.8%** | 0.0001 |
| 9 | 1000 | ug | FGF1 | 4 | hr | 28.72 ± 11.48 | −64.4%** | 0.0001 |
| 10 | 1000 | ug | FGF1 | 8 | hr | 64.58 ± 16.68 | −17.4%** | NS (0.1085) |
| 11 | 1000 | ug | FGF1 | 12 | hr | 74.55 ± 11.41 | −6.3%** | NS (0.6752) |
| 12 | 1000 | ug | FGF1 | 24 | hr | 79.96 ± 11.72 | −8.9%** | NS (0.2362) |

Percent decreases are compared to the respective vehicle control animals.

*Compared to Group 1 (Vehicle)
**Compared to appropriate vehicle group

Mortality: There were several deaths in this study (Table 2). The animal deaths were attributed to problems associated with the ischemic injury paradigm. We added animals to the groups to bring the final number of animals per group to 10.

TABLE 2
Number of deaths per group.

| Group | | Compound | Dose (mg/kg) | Deaths |
|---|---|---|---|---|
| 1 | (n = 10 mice) | Vehicle | 0 ug/kg/hr × 3 hrs start at 1 hr | 0 |
| 2 | (n = 10 mice) | Vehicle | 0 ug/kg/hr × 3 hrs start at 2 hr | 0 |
| 3 | (n = 11 mice) | Vehicle | 0 ug/kg/hr × 3 hrs start at 4 hr | 1 |
| 4 | (n = 12 mice) | Vehicle | 0 ug/kg/hr × 3 hrs start at 8 hr | 2 |
| 5 | (n = 12 mice) | Vehicle | 0 ug/kg/hr × 3 hrs start at 12 hr | 2 |
| 6 | (n = 12 mice) | Vehicle | 0 ug/kg/hr × 3 hrs start at 24 hr | 2 |
| 7 | (n = 10 mice) | FGF-1 | 1000 ug/kg/hr × 3 hrs start at 1 hr | 0 |
| 8 | (n = 10 mice) | FGF-1 | 1000 ug/kg/hr × 3 hrs start at 2 hr | 0 |
| 9 | (n = 10 mice) | FGF-1 | 1000 ug/kg/hr × 3 hrs start at 4 hr | 0 |
| 10 | (n = 11 mice) | FGF-1 | 1000 ug/kg/hr × 3 hrs start at 8 hr | 1 |
| 11 | (n = 10 mice) | FGF-1 | 1000 ug/kg/hr × 3 hrs start at 12 hr | 0 |
| 12 | (n = 11 mice) | FGF-1 | 1000 ug/kg/hr × 3 hrs start at 12 hr | 1 |

Behavioral Measurements. Animals were assessed for neurological deficits based on a scale of 0 to 4. Animals treated with FGF-1 showed a time dependent decrease in neurological deficits (FIG. 2 and Table 3).

TABLE 3
Neurological deficits following FGF treatment

| Compound | Neurological Deficits |
|---|---|
| Vehicle (1 hr) | 3.20 ± 0.789 (NA) |
| Vehicle (2 hr) | 3.10 ± 0.738 (NS)* |
| Vehicle (4 hr) | 3.10 ± 0.876 (NS)* |
| Vehicle (8 hr) | 3.20 ± 0.789 (NS)* |
| Vehicle (12 hr) | 3.20 ± 0.919 (NS)* |
| Vehicle (24 hr) | 3.70 ± 0.675 (NS)* |
| FGF-1 (1000 ug) (1 hr) | 3.70 ± 0.483 (0.0001)* |
| FGF-1 (1000 ug) (2 hr) | 1.00 ± 0.471 (0.0001)** |
| FGF-1 (1000 ug) (4 hr) | 1.60 ± 0.516 (0.0002)** |
| FGF-1 (1000 ug) (8 hr) | 2.30 ± 0.823 (0.0225)** |
| FGF-1 (1000 ug) (12 hr) | 2.90 ± 0.738 (NS)** |
| FGF-1 (1000 ug) (24 hr) | 3.20 ± 0.633 (NS)** |

*compared to Vehicle (1 hr)
**compared to appropriate vehicle control

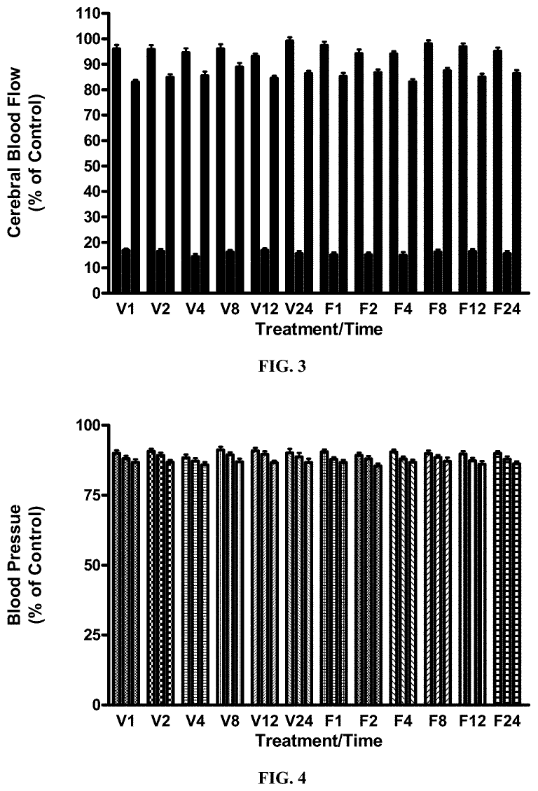

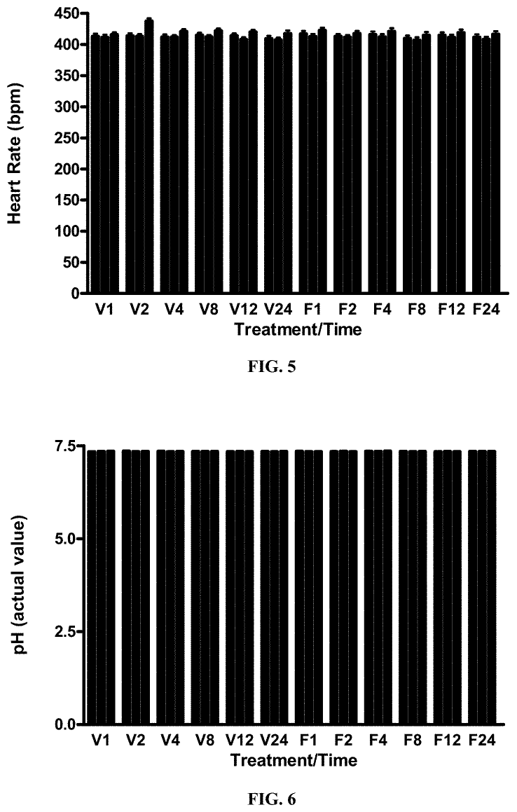

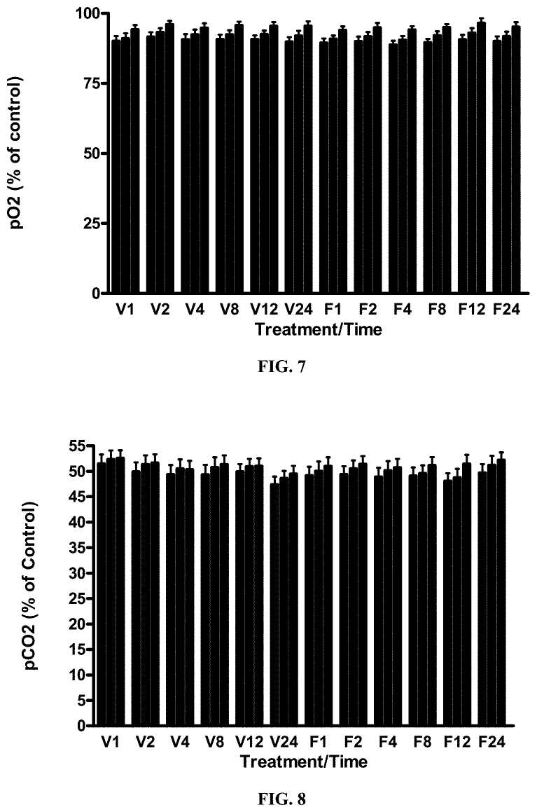

Physiological parameters. There were no significant differences in physiological parameters [cerebral blood flow (FIG. 3), mean arterial pressure (FIG. 4), heart rate (FIG. 5), blood pH (FIG. 6), pO₂ (FIG. 7), and pCO₂ (FIG. 8)) between the vehicle and treated mice at baseline, during ischemia, or after reperfusion (Table 4).

TABLE 4
Physiological parameters in vehicle and FGF treated mice
at baseline, during ischemia, and after reperfusion.

| | Pre-ischemia | Ischemia | Post-ischemia |
|---|---|---|---|
| Cerebral Blood Flow | | | |
| (% of baseline) | | | |
| Vehicle (1 hr) | 96.1 ± 1.464 | 16.8 ± 0.800 | 83.0 ± 0.882 |
| Vehicle (2 hr) | 95.9 ± 1.602 | 16.5 ± 0.946 | 84.9 ± 1.197 |
| Vehicle (4 hr) | 94.6 ± 1.688 | 14.4 ± 1.013 | 85.5 ± 1.662 |
| Vehicle (8 hr) | 96.1 ± 1.779 | 16.2 ± 0.786 | 88.9 ± 1.622 |
| Vehicle (12 hr) | 93.2 ± 1.052 | 16.8 ± 1.867 | 84.5 ± 1.014 |
| Vehicle (24 hr) | 99.2 ± 1.420 | 15.6 ± 1.002 | 86.4 ± 1.035 |
| FGF-1 (1 hr) | 97.4 ± 1.485 | 15.1 ± 0.971 | 85.3 ± 1.300 |
| FGF-1 (2 hr) | 94.2 ± 1.569 | 15.1 ± 0.912 | 86.7 ± 1.239 |
| FGF-1 (4 hr) | 94.1 ± 1.016 | 14.9 ± 1.251 | 83.1 ± 1.080 |
| FGF-1 (8 hr) | 98.1 ± 1.303 | 16.2 ± 0.975 | 87.5 ± 1.088 |
| FGF-1 (12 hr) | 96.9 ± 1.337 | 16.4 ± 0.945 | 85.0 ± 1.291 |
| FGF-1 (24 hr) | 95.1 ± 1.456 | 15.6 ± 1.013 | 86.4 ± 1.275 |
| Blood Pressure | | | |
| (mmHg) | | | |
| Vehicle (1 hr) | 90.06 ± 1.023 | 88.10 ± 1.015 | 86.75 ± 1.126 |
| Vehicle (2 hr) | 90.69 ± 0.990 | 89.26 ± 1.012 | 86.86 ± 0.809 |
| Vehicle (4 hr) | 88.41 ± 1.171 | 87.22 ± 1.046 | 85.85 ± 0.985 |
| Vehicle (8 hr) | 91.23 ± 1.088 | 89.46 ± 0.946 | 86.96 ± 1.144 |
| Vehicle (12 hr) | 90.89 ± 1.053 | 89.67 ± 1.020 | 86.63 ± 0.767 |
| Vehicle (24 hr) | 90.13 ± 1.440 | 88.72 ± 1.442 | 86.73 ± 1.369 |
| FGF-1 (1 hr) | 90.53 ± 0.878 | 87.86 ± 0.734 | 86.74 ± 0.928 |
| FGF-1 (2 hr) | 89.28 ± 0.960 | 88.05 ± 0.979 | 85.45 ± 0.939 |
| FGF-1 (4 hr) | 90.47 ± 0.859 | 87.95 ± 0.925 | 86.78 ± 0.915 |
| FGF-1 (8 hr) | 89.92 ± 1.036 | 88.55 ± 0.844 | 87.14 ± 1.344 |
| FGF-1 (12 hr) | 89.81 ± 0.959 | 87.33 ± 1.043 | 86.10 ± 1.104 |
| FGF-1 (24 hr) | 89.96 ± 0.800 | 87.90 ± 1.023 | 86.25 ± 0.876 |
| Heart Rate (bpm)* | | | |
| Vehicle (1 hr) | 413.2 ± 4.240 | 411.7 ± 4.096 | 416.0 ± 3.357 |
| Vehicle (2 hr) | 414.1 ± 4.097 | 413.2 ± 3.533 | 437.9 ± 4.212 |
| Vehicle (4 hr) | 412.3 ± 3.807 | 412.1 ± 3.000 | 421.2 ± 3.470 |
| Vehicle (8 hr) | 415.4 ± 3.474 | 412.5 ± 2.701 | 422.3 ± 3.426 |
| Vehicle (12 hr) | 414.3 ± 3.627 | 408.3 ± 3.208 | 420.2 ± 3.087 |
| Vehicle (24 hr) | 409.7 ± 4.185 | 407.7 ± 3.370 | 417.9 ± 4.550 |
| FGF-1 (1 hr) | 417.3 ± 4.556 | 413.0 ± 3.978 | 422.9 ± 4.223 |
| FGF-1 (2 hr) | 413.2 ± 3.663 | 411.8 ± 3.593 | 417.7 ± 4.020 |
| FGF-1 (4 hr) | 416.0 ± 4.773 | 412.4 ± 4.375 | 421.2 ± 5.164 |
| FGF-1 (8 hr) | 410.0 ± 4.427 | 407.1 ± 4.486 | 415.1 ± 4.927 |
| FGF-1 (12 hr) | 414.9 ± 4.518 | 411.6 ± 4.116 | 419.1 ± 4.900 |
| FGF-1 (24 hr) | 411.8 ± 4.718 | 408.6 ± 3.868 | 416.6 ± 4.992 |
| pO₂ (mmHg) | | | |
| Vehicle (1 hr) | 90.12 ± 1.763 | 91.03 ± 1.856 | 94.19 ± 1.624 |
| Vehicle (2 hr) | 91.57 ± 1.724 | 93.22 ± 1.529 | 96.04 ± 1.300 |
| Vehicle (4 hr) | 90.64 ± 1.937 | 92.43 ± 1.808 | 94.79 ± 1.690 |
| Vehicle (8 hr) | 90.73 ± 1.660 | 92.41 ± 1.551 | 95.69 ± 1.351 |
| Vehicle (12 hr) | 90.73 ± 1.447 | 92.53 ± 1.326 | 95.47 ± 1.424 |
| Vehicle (24 hr) | 89.84 ± 1.686 | 91.95 ± 1.811 | 95.46 ± 1.720 |
| FGF-1 (1 hr) | 89.45 ± 1.582 | 90.82 ± 1.296 | 93.98 ± 1.384 |
| FGF-1 (2 hr) | 89.99 ± 1.743 | 91.70 ± 1.656 | 94.91 ± 1.708 |
| FGF-1 (4 hr) | 88.78 ± 1.464 | 90.44 ± 1.467 | 94.10 ± 1.330 |
| FGF-1 (8 hr) | 89.58 ± 1.314 | 92.08 ± 1.486 | 94.95 ± 1.206 |
| FGF-1 (12 hr) | 90.67 ± 1.675 | 92.95 ± 1.744 | 96.56 ± 1.732 |
| FGF-1 (24 hr) | 90.06 ± 1.715 | 91.80 ± 1.690 | 95.18 ± 1.678 |
| pCO₂ (mmHg) | | | |
| Vehicle (1 hr) | 51.46 ± 1.848 | 52.32 ± 1.767 | 52.57 ± 1.559 |
| Vehicle (2 hr) | 49.94 ± 1.821 | 51.34 ± 1.795 | 51.65 ± 1.666 |
| Vehicle (4 hr) | 49.38 ± 1.842 | 50.53 ± 1.793 | 53.33 ± 1.738 |
| Vehicle (8 hr) | 49.34 ± 1.930 | 50.77 ± 1.992 | 51.33 ± 1.803 |
| Vehicle (12 hr) | 49.94 ± 1.456 | 50.93 ± 1.512 | 51.02 ± 1.535 |
| Vehicle (24 hr) | 47.37 ± 1.592 | 48.64 ± 1.420 | 49.48 ± 1.576 |
| FGF-1 (1 hr) | 49.17 ± 1.708 | 50.04 ± 1.876 | 50.99 ± 1.749 |
| FGF-1 (2 hr) | 49.37 ± 1.612 | 50.51 ± 1.620 | 51.40 ± 1.603 |
| FGF-1 (4 hr) | 48.91 ± 1.781 | 50.12 ± 1.864 | 50.72 ± 1.719 |
| FGF-1 (8 hr) | 49.90 ± 1.681 | 49.57 ± 1.601 | 51.18 ± 1.608 |
| FGF-1 (12 hr) | 48.07 ± 1.552 | 48.76 ± 1.724 | 51.43 ± 1.800 |
| FGF-1 (24 hr) | 49.68 ± 1.734 | 51.21 ± 1.864 | 52.20 ± 1.500 |
| pH | | | |
| Vehicle (1 hr) | 7.340 ± 0.006 | 7.348 ± 0.005 | 7.354 ± 0.006 |
| Vehicle (2 hr) | 7.356 ± 0.005 | 7.342 ± 0.004 | 7.348 ± 0.004 |
| Vehicle (4 hr) | 7.351 ± 0.007 | 7.342 ± 0.006 | 7.346 ± 0.006 |
| Vehicle (8 hr) | 7.350 ± 0.009 | 7.349 ± 0.008 | 7.345 ± 0.004 |
| Vehicle (12 hr) | 7.342 ± 0.009 | 7.346 ± 0.008 | 7.345 ± 0.009 |
| Vehicle (24 hr) | 7.350 ± 0.007 | 7.344 ± 0.008 | 7.350 ± 0.009 |
| FGF-1 (1 hr) | 7.351 ± 0.007 | 7.342 ± 0.008 | 7.343 ± 0.007 |
| FGF-1 (2 hr) | 7.344 ± 0.011 | 7.351 ± 0.006 | 7.342 ± 0.008 |
| FGF-1 (4 hr) | 7.353 ± 0.008 | 7.346 ± 0.007 | 7.359 ± 0.008 |
| FGF-1 (8 hr) | 7.348 ± 0.012 | 7.344 ± 0.008 | 7.347 ± 0.010 |
| FGF-1 (12 hr) | 7.341 ± 0.008 | 7.346 ± 0.006 | 7.345 ± 0.007 |
| FGF-1 (24 hr) | 7.347 ± 0.009 | 7.349 ± 0.008 | 7.347 ± 0.007 |

BPM, beats per minute

Infusion of vehicle at various times (1, 2, 4, 8, 12, 24 hrs) demonstrated the typical infarct volume in the mouse model of ischemia. There was no difference in the infarct volume with administration of vehicle at any time. Compared to the vehicle, there were significant effects of FGF-1 on infarct volume at the different times of administration. FGF-1 showed significant protection in this model at 1, 2, and 4 hrs with a decrease at 8, 12 and 24 but these did not reach the level of significance. Based on these data FGF-1 appears to be effective in the mouse model of cerebral ischemia. The clinically relevant approach in this study is the delivery of the drugs via i.v. infusion following the injury for up to 4 hrs to determine the beneficial effects of the test articles. Under these parameters FGF-1 showed a greater protection due to the access to the brain tissue following injury. In addition, these studies suggest that FGF-1 has a therapeutic window of opportunity up to 4-8 hrs following injury.

These data show that the FGF-1, FGF-1₁₋₁₅₅, FGF-1₁₋₁₄₁, or combinations thereof are effective in the treatment of stroke. FGF-1, FGF-1₁₋₁₅₅, FGF-1₁₋₁₄₁, or combinations thereof being a FGF compounds most likely bind to the receptors in vivo and protect against neuronal cell death. The FGF compounds possibly bind to their cognate receptor and interfere with molecules interacting in the brain to cause insult.

When administered by an i.v. infusion, that FGF-1, FGF-1₁₋₁₅₅, FGF-1₁₋₁₄₁, or combinations thereof, are effective to be protective in the mouse model of cerebral ischemia. FGF-1 was a relatively potent protective agent in mice against cerebral ischemia and reperfusion injury even when the initiation of therapy was started 2-4 hrs after injury (statistically significant at p<0.0001, indicating that there was a significant protection for ischemic injury). In addition, FGF-1 was significantly effective at time points when initiated at 4 hrs and possibly 8 hrs after injury.

### Example 2. Treatment of Animal Stroke Models with FGF-1₁₋₁₅₅, FGF-1₁₋₁₄₁, or Combinations Thereof

Stroke resulting in brain damage is most often caused by a lack of blood flow to a selected part of the brain. A stroke results in permanent damage to the brain tissue and in many cases, permanent disability to the patient. Stroke is the third leading cause of death and a leading cause of serious, long-term disability in the United States. The probability of stroke increases as people get older. According to the American Heart Association, approximately 700,000 Americans suffer a stroke each year; about 25% of these strokes are fatal. Stroke is responsible for an estimated $40 billion in health-care costs and lost productivity each year.

Ischemic or occlusive strokes, which account for approximately 80 percent of all strokes, are caused by an obstruction in an artery, generally one of the neck carotid arteries, the major arteries in the neck that carry oxygen-rich blood from the heart to the brain. There is limited treatment available to patients who have suffered a stroke. Thrombolytic therapy using plasminogen activators is sometimes tried in these patients to unblock the arteries supplying blood to the brain, but safety and bleeding issues have prevented this treatment from gaining wide-spread acceptance in the medical community.

Animal studies have shown the potential of growth factor therapy in limiting the severity of brain ischemia after a stroke. A stroke is characterized by an infarcted area of the brain, dead tissue, which cannot recover, surrounded by an underperfused area of risk, which would be the target of growth factor treatment.

Studies in Animal Stroke Models. The inventors tested FGF-1₁₋₁₅₅, FGF-1₁₋₁₄₁, or combinations thereof in an animal model of stroke. In this model, the mice are given an experimental stroke by blocking the flow of blood into the brain for 1 hr after which either animals were dosed by I.V. infusion with control or FGF-1₁₋₁₅₅, FGF-1₁₋₁₄₁, or combinations thereof for 3 hrs. The volume of the stroke is measured, and behavioral tests that indicate the degree of neurological deficits in the animals after 24 hrs are also performed. The first study in mice tested FGF-1₁₋₁₅₅, FGF-1₁₋₁₄₁, or combinations thereof at doses of 20, 50 and 100 ug/kg/hr. The 50 ug/kg/hr dose was protective, and the 100 ug/kg/hr dose was highly protective. A second trial in which mice received increasing doses of FGF-1₁₋₁₅₅, FGF-1₁₋₁₄₁, or combinations thereof of 200, 500 and 1000 ug/kg/hr has also been completed. There was a dose-dependent decrease in the volume of the stroke area as more FGF-1₁₋₁₅₅, FGF-1₁₋₁₄₁, or combinations thereof was given to the animals. The 1000 ug/kg/hr dose group displayed stroke volumes that were decreased by over 80% when compared to control animals. Also seen in these studies was a significant improvement in neurological defects as the dose of FGF-1₁₋₁₅₅, FGF-1₁₋₁₄₁, or combinations thereof increased. Graphs of the results from these animal studies are shown below.

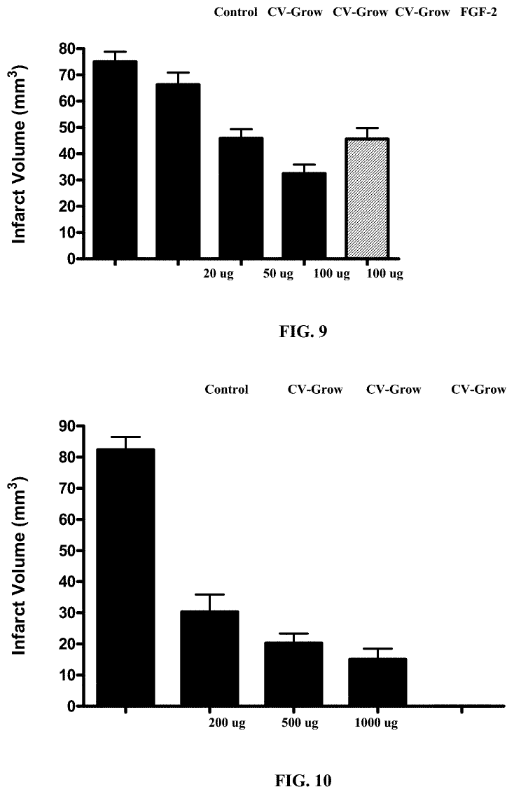

FIG. 9 shows the results from a study in mice with Experimental Stroke: Effect of FGF-1₁₋₁₅₅, FGF-1₁₋₁₄₁, or combinations thereof on Infarct Volumes in the Mouse Following Transient Ischemia. All mice were subjected to 1 hr of cerebral ischemia followed by 24 hrs of reperfusion. Animals were infused with vehicle (control), FGF-1₁₋₁₅₅, FGF-1₁₋₁₄₁, or combinations thereof or FGF-2 intravenously at the end of ischemia for 3 hrs. Animals were sacrificed on day 2 and processed to determine the infarct volume. FGF-2, or basic FGF, is another member of the fibroblast growth factor family and has previously been shown to be efficacious in this animal model of stroke.

FIG. 10 shows the results from a study in mice with Experimental Stroke: FGF-1₁₋₁₅₅, FGF-1₁₋₁₄₁, or combinations thereof on Infarct Volumes in the Mouse Following Transient Ischemia. All mice were subjected to 1 hr of cerebral ischemia followed by 24 hrs of reperfusion. Animals were infused with vehicle (control), FGF-1₁₋₁₅₅, FGF-1₁₋₁₄₁, or combinations thereof or FGF-2 intravenously at the end of ischemia for 3 hrs. Animals were sacrificed on day 2 and processed to determine the infarct volume. FGF-2 was not tested in this series of experiments.

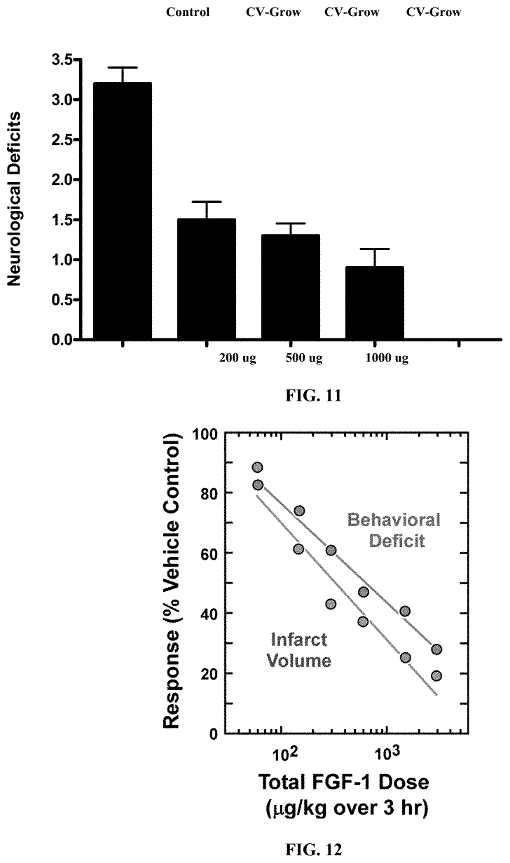

FIG. 11 shows the effect of FGF-1₁₋₁₅₅, FGF-1₁₋₁₄₁, or combinations thereof on Neurological Deficits in the Mouse Following Transient Ischemia. All mice were subjected to 1 hr of cerebral ischemia followed by infusion of FGF-1₁₋₁₅₅, FGF-1₁₋₁₄₁, or combinations thereof. Animals were examined for neurological deficits at 22 hrs after ischemia.

FIG. 12 shows that the size of stroke volume correlates with behavioral deficits. From the above figure, it can be seen that there is a very tight correlation between the size of the stroke volume and the degree of behavioral deficits in mice given a stroke. By reducing the volume of stroke, FGF-1₁₋₁₅₅, FGF-1₁₋₁₄₁, or combinations thereof significantly reduces behavioral deficits.

These studies demonstrate the use of FGF-1₁₋₁₅₅, FGF-1₁₋₁₄₁, or combinations thereof to dramatically decrease the area of a stroke in experimental animal models. In a dose-dependent fashion, FGF-1₁₋₁₅₅, FGF-1₁₋₁₄₁, or combinations thereof decreased the volume of a stroke to a statistically significant extent over control animals. At the highest dose of FGF-1₁₋₁₅₅, FGF-1₁₋₁₄₁, or combinations thereof over an 80% decrease in the volume of the stroke area was noted with a significant increase in the neurological functioning of these animals as assessed by behavioral studies.

There is currently no treatment in the marketplace that has widespread acceptance for the treatment of stroke. The debilitating effects that patients suffer following a stroke, as well as the expensive medical treatment needed to care for stroke patients, is a primary motivator for the development of FGF-1₁₋₁₅₅, FGF-1₁₋₁₄₁, or combinations thereof as therapeutics.

### Example 3. Intravenous Administration of FGF-1

Infusion of vehicle demonstrated the typical infarct volume in the mouse model of ischemia. Compared to the vehicle, there were significant effects of FGF-1 on infarct volumes. FGF-1 showed protection in this model. In addition, FGF-1 at 1000 mg/kg/hr dose was the most effective at protecting the brain from injury. Based on these data, the test articles appear to be effective in the model. The clinically relevant approach in this study is the delivery of the drugs via intravenous (i.v.) infusion following the injury for up to 3 hrs to determine the beneficial effects of the test articles. Under these parameters the test article showed a greater protection due to the access to the brain tissue following injury.

These data show that the FGF-1 is effective in the treatment of stroke. The FGF-1 most likely binds to the receptors in vivo and protect against neuronal cell death. The FGF compounds bind to their cognate receptor and interfere with molecules interacting in the brain to cause insult. These results show the effect of FGF-1 in a model system for human stroke.

When administered by an i.v. infusion, FGF-1 is shown to be protective in the mouse model of cerebral ischemia. FGF-1 was a relatively potent protective agent in mice against cerebral ischemia and reperfusion injury (statistically significant at p<0.0001, indicating that there was a significant protection for ischemic injury). In addition, FGF-1 was significantly effective at all doses but most effective as 1000 μg/kg/hr (p<0.0001).

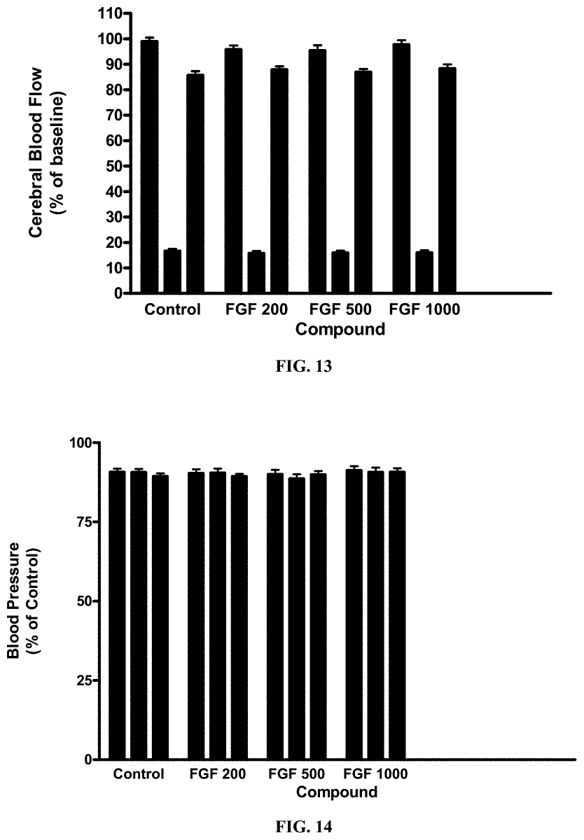

FIG. 13 is a graph that shows the effect of FGF-1 on cerebral blood flow in the mouse following transient ischemia. All mice were subjected to 1 hr of cerebral ischemia followed by 24 hrs of reperfusion. Animals were examined for CBF at 10 min prior to, during and after ischemia.

FIG. 14 is a graph that shows the effect of FGF-1 on blood pressure in the mouse following transient ischemia. All mice were subjected to 1 hr of cerebral ischemia followed by 24 hrs of reperfusion. Animals were examined for BP at 10 min prior to, during and after ischemia.

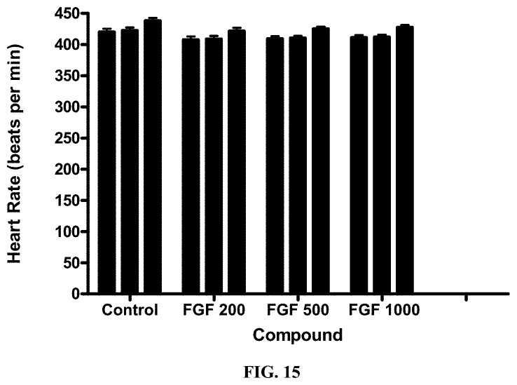

FIG. 15 is a graph that shows the effect of FGF-1 on heart rate in the mouse following transient ischemia. All mice were subjected to 1 hr of cerebral ischemia followed by 24 hrs of reperfusion. Animals were examined for HR at 10 min prior to, during and after ischemia.

In yet another embodiment, the present invention includes providing the FGF-1 prophylactically, prior to a surgery in which ischemia is possible or planned. It was found that the present invention was protective in the mouse model of cerebral ischemia.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Bean A J, Elde R, Cao Y H, Oellig C, Tamminga C, Goldstein M, Petterson R F, Hokfelt T (1991) Expression of acidic and basic fibroblast growth factors in the substantia nigra of rat. Monkey and human. Proc. Natl. Acad. Sci. 88:10237-10241.
Bethel A, Kirsch J R, Koelher R C, Finkelstein S P, Traystman R J (1997) Intravenous basic fibroblast growth factor decreases brain injury resulting from focal ischemia in cats. Stroke 28:609-615.
Bieger S, Unsicker K (1996) Functions of fibroblast growth factors (FGFs) in the nervous system. In: Bell C (ed) Chemical factors in neural growth, degeneration and repair. Elsevier Science BV.
Mufson E J, Kronin J S, Sendera T J, Sobreviela T (1999) Distribution and retrograde transport of trophic factors in the central nervous system: functional implications for the treatment of neurodegenerative diseases. Prog. Neurobiol. 57:451-484.
Nakata N, Kato H, Kogure K (1993) Protective effects of basic fibroblast growth factor against hippocampal neuronal damage following cerebral ischemia in the gerbil. Brain Res. 605:354-356.
Ogata N, Matsushima M, Takada Y, Tobe T, Takahashi K, Yi X, Yamamoto C, Yamada H, Uyuma M (1996) Expression of basic fibroblast growth factor mRNA in developing chorodial neovascularization. Curr Eye Res 15:1008-1018.
Patel M N, McNamara J O (1995) Selective enhancement of axonal branching of cultured dentate gyrus neurons by neurotrophic factors. Neuroscience. 69:763-770.
Sasaki K, Oomura Y, Suzuki K, Hanai K, Yagi H (1992) Acidic fibroblast growth factor prevents death of hippocampal CA1 pyramidal cells following ischemia. Neurochem Int 21:397-402.
Vaccarino F M, Schwartz M L, Raballo R, Rhee H, Lyn-Cook R (1999) Fibroblast growth factor signaling regulates growth and morphogenesis at multiple steps during brain development. Curr. Top Dev Biol 46:179-200.
Vaccarino F M, Ganat Y, Zhang Y, Zheng W (2001) Stem cells in neurodevelopment and plasticity. Neuropsychopharmacology 25:805-815.
Vicario-Abejon C, Johe K K, Hazel T G, Collazo D, McKay R D (1995) Functions of basic fibroblast growth factor and neurotrophins in the differentiation of hippocampal neurons. Neuron 15:105-114.

What is claimed is:

1. A method of treating ischemic stroke, comprising administering to a subject with ischemic stroke an FGF-1, FGF-1₁₋₁₅₅, FGF-1₁₋₁₄₁, or combinations thereof, in an amount sufficient to cross the blood brain barrier and treat the ischemic stroke.

2. The method of claim 1, further comprising administering at least one other therapeutic agent to the subject, before, concurrently with or after the FGF-1, FGF-1₁₋₁₅₅, FGF-1₁₋₁₄₁, or combinations thereof.

3. The method of claim 2, wherein the therapeutic agent is selected from the group consisting of a second antibody, a second antibody fragment, an immunoconjugate, an immunomodulator, an anti-angiogenic agent, a pro-apoptotic agent, a cytokine, a chemokine, a drug, a hormone, an siRNA, a coagulation inhibitor, a stem cell growth factor, a lymphotoxin, a hematopoietic factor, a colony stimulating factor, an interferon, erythropoietin, thrombopoietin, an enzyme, recombinant human thrombomodulin and activated human protein C.

4. The method of claim 3, wherein the FGF-1, FGF-1₁₋₁₅₅, FGF-1₁₋₁₄₁, or combinations thereof, are provided intravenously, subcutaneously, intranasally, stereotaxically delivered into a brain parenchyma, into the cerebrospinal fluid, or in an indwelling Ommaya reservoir.

5. The method of claim 1, wherein a brain image of the subject is captured in an ambulance and administration of the FGF-1, FGF-1₁₋₁₅₅, FGF-1₁₋₁₄₁, or combinations thereof, occurs before arrival to a hospital.

6. The method of claim 1, wherein the subject has been administered tissue plasminogen activator after the subject has suffered the cerebral ischemia/reperfusion injury.

7. The method of claim 1, wherein the FGF-1, FGF-1₁₋₁₅₅, FGF-1₁₋₁₄₁, or combinations thereof, is comprised within a pharmaceutical composition formulated for injection, or for sustained release.

8. The method of claim 1, wherein the FGF-1, FGF-1₁₋₁₅₅, FGF-1₁₋₁₄₁, or combinations thereof, is administered to the subject within 24 hrs of the onset of symptoms of ischemic stroke.

9. The method of claim 1, wherein the FGF-1, FGF-1₁₋₁₅₅, FGF-1₁₋₁₄₁, or combinations thereof, is repeatedly administered to the subject at least once per day for at least 3 days.

10. The method of claim 1, wherein the FGF-1, FGF-1₁₋₁₅₅, FGF-1₁₋₁₄₁, or combinations thereof, is repeatedly administered to the subject for at least 7 days.

11. The method of claim 1, wherein the FGF-1, FGF-1₁₋₁₅₅, FGF-1₁₋₁₄₁, or combinations thereof, is provided at 20, 50, 100, 200, 500 and 1000 ug/kg/hr.

12. The method of claim 1, wherein the FGF-1, FGF-1₁₋₁₅₅, FGF-1₁₋₁₄₁, or combinations thereof, reduces an infarct volume by more than 25, 30, 33, 35, 40, 45, or 50% when compared to a non-treated tissue.

13. The method of claim 1, wherein the FGF-1, FGF-1₁₋₁₅₅, FGF-1₁₋₁₄₁, or combinations thereof, reduces a neurological deficit by at least 30, 40, 50, 60, or 70% when compared to a non-treated tissue.

14. The method of claim 1, wherein a first injection site is within the ischemic region of brain tissue.

15. The method of claim 1, wherein a first injection site is directly adjacent to the ischemic region of brain tissue.

16. The method of claim 1, wherein a first injection site is outside of the ischemic region of brain tissue.

17. The method of claim 1, further comprising: obtaining a preoperative non-invasive image data of the subject, the preoperative non-invasive image data including a region of brain tissue, analyzing the preoperative non-invasive image data to preoperatively identify at least one at-risk region of brain tissue, preoperatively identifying at least one abnormality within a blood vessel supply to the at-risk region of brain tissue, operatively administering a therapeutically effective amount of the FGF-1, FGF-1₁₋₁₅₅, FGF-1₁₋₁₄₁, or combinations thereof, wherein the administration of the FGF-1, FGF-1₁₋₁₅₅, FGF-1₁₋₁₄₁, or combinations thereof induces growth of supplemental blood vessels proximate to the abnormality.

18. The method of claim 1, further comprising the steps of obtaining postoperative non-invasive image data of the subject, the post-operative non-invasive image data including the at-risk region of brain tissue, analyzing the post-operative non-invasive image data to identify any improvement in the blood vessel supply to the at-risk region of brain tissue.

19. A method of treating ischemic stroke, comprising:
identifying a subject in need of treatment for ischemic stroke; and
administering to a subject with ischemic stroke an FGF-1, FGF-1₁₋₁₅₅, FGF-1₁₋₁₄₁, or combinations thereof, in an amount sufficient to cross the blood-brain barrier.

20. The method of claim 19, further comprising administering at least one other therapeutic agent to the subject, before, concurrently with or after the FGF-1, FGF-1₁₋₁₅₅, FGF-1₁₋₁₄₁, or combinations thereof.

21. The method of claim 20, wherein the therapeutic agent is selected from the group consisting of a second antibody, a second antibody fragment, an immunoconjugate, an immunomodulator, an anti-angiogenic agent, a pro-apoptotic agent, a cytokine, a chemokine, a drug, a hormone, an siRNA, a coagulation inhibitor, a stem cell growth factor, a lymphotoxin, a hematopoietic factor, a colony stimulating factor, an interferon, erythropoietin, thrombopoietin, an enzyme, recombinant human thrombomodulin and activated human protein C.

22. The method of claim 21, wherein the FGF-1, FGF-1₁₋₁₅₅, FGF-1₁₋₁₄₁, or combinations thereof, are provided intravenously, subcutaneously, intranasally, stereotaxically delivered into a brain parenchyma, into the cerebrospinal fluid, or in an indwelling Ommaya reservoir.

23. The method of claim 19, wherein a brain image of the subject is captured in an ambulance and administration of the FGF-1, FGF-1₁₋₁₅₅, FGF-1₁₋₁₄₁, or combinations thereof, occurs before arrival to a hospital.

24. The method of claim 19, wherein the subject has been administered tissue plasminogen activator after the subject has suffered the cerebral ischemia/reperfusion injury.

25. The method of claim 19, wherein the FGF-1, FGF-1₁₋₁₅₅, FGF-1₁₋₁₄₁, or combinations thereof, is comprised within a pharmaceutical composition formulated for injection or sustained release.

26. The method of claim 19, wherein the FGF-1, FGF-1₁₋₁₅₅, FGF-1₁₋₁₄₁, or combinations thereof, is administered to the subject within 24 hrs of the onset of symptoms of ischemic stroke.

27. The method of claim 19, wherein the FGF-1, FGF-1₁₋₁₅₅, FGF-1₁₋₁₄₁, or combinations thereof, is repeatedly administered to the subject at least once per day for at least 3 days.

28. The method of claim 19, wherein the FGF-1, FGF-1₁₋₁₅₅, FGF-1₁₋₁₄₁, or combinations thereof, is repeatedly administered to the subject for at least 7 days.

29. The method of claim 19, wherein the FGF-1, FGF-1₁₋₁₅₅, FGF-1₁₋₁₄₁, or combinations thereof, is provided at 20, 50, 100, 200, 500 and 1000 ug/kg/hr.

30. The method of claim 19, wherein the FGF-1, FGF-1₁₋₁₅₅, FGF-1₁₋₁₄₁, or combinations thereof, reduces an infarct volume by more than 25, 30, 33, 35, 40, 45, or 50% when compared to a non-treated tissue.

31. The method of claim 19, wherein the FGF-1, FGF-1₁₋₁₅₅, FGF-1₁₋₁₄₁, or combinations thereof, reduces a neurological deficit by at least 30, 40, 50, 60, or 70% when compared to a non-treated tissue.

32. The method of claim 19, further comprising: obtaining a preoperative non-invasive image data of the subject, the preoperative non-invasive image data including a region of brain tissue, analyzing the preoperative non-invasive image data to preoperatively identify at least one at-risk region of brain tissue, preoperatively identifying at least one abnormality within a blood vessel supply to the at-risk region of brain tissue, operatively administering a therapeutically effective amount of the FGF-1, FGF-1₁₋₁₅₅, FGF-1₁₋₁₄₁, or combinations thereof, wherein the administration of the FGF-1, FGF-1₁₋₁₅₅, FGF-1₁₋₁₄₁, or combinations thereof induces growth of supplemental blood vessels proximate to the abnormality.

33. The method of claim 19, further comprising administering the FGF-1, FGF-1₁₋₁₅₅, FGF-1₁₋₁₄₁, or combinations thereof to induce growth of blood vessels proximate to the abnormality, wherein at least a portion of the blood vessel supply to the at-risk region of brain tissue is redirected through the supplemental blood vessels.

34. The method of claim 19, wherein the abnormality is located within the at-risk region of brain tissue.

35. The method of claim 19, wherein the abnormality is located within the region of brain tissue.

36. The method of claim 19, further comprising the steps of obtaining postoperative non-invasive image data of the subject, the post-operative non-invasive image data including the at-risk region of brain tissue, analyzing the post-operative non-invasive image data to identify any improvement in the blood vessel supply to the at-risk region of brain tissue.

37. The method of claim 19, wherein a first injection site is within the ischemic region of brain tissue.

38. The method of claim 19, wherein a first injection site is directly adjacent to the ischemic region of brain tissue.

39. The method of claim 19, wherein a first injection site is outside of the ischemic region of brain tissue.
